(19) United States
(12) Patent Application Publication
(10) Pub. No.: US 2026/0212957 A1
(43) Pub. Date: Jul. 23, 2026

(54) DIFFUSION MODEL FOR PROTEIN-PROTEIN MODELING

(71) Applicant: NEC Laboratories America, Inc., Princeton, NJ (US)

(72) Inventors: Renqiang Min, Princeton, NJ (US); Zhenqiao Song, Pittsburgh, PA (US); Tianxiao Li, Plainsboro, NJ (US)

(21) Appl. No.: 19/453,396

(22) Filed: Jan. 20, 2026

Related U.S. Application Data
(60) Provisional application No. 63/748,179, filed on Jan. 22, 2025.
(60) Provisional application No. 63/802,763, filed on May 9, 2025.
(60) Provisional application No. 63/841,676, filed on Jul. 10, 2025.

(51) Int. Cl. G16B 40/00; G01N 33/68

(52) U.S. Cl. CPC G16B 40/00; G01N 33/68

(57) ABSTRACT

Methods and systems for protein generation include determining a binding protein that binds with a target protein using a hybrid diffusion model that jointly generates a discrete residue sequence and a continuous backbone structure for the binding protein. The binding protein is manufactured.

### RELATED APPLICATION INFORMATION

This application claims priority to U.S. Patent Application No. 63/748,179, filed on Jan. 22, 2025, to U.S. Patent Application No. 63/802,763, filed on May 9, 2025, and to U.S. Patent Application No. 63/841,676, filed on Jul. 10, 2025, each incorporated herein by reference in its entirety.

### BACKGROUND

### Technical Field

The present invention relates to protein design and, more particularly, to the use of diffusion models for protein design.

### Description of the Related Art

Biomedicine makes use of proteins that are designed with high affinity and specificity for protein targets of interest. Applications include therapeutic development, diagnostics, and imaging reagents. Existing approaches to design proteins require considerable experimental effort, which is costly in both time and money. Computational approaches are challenged by proteins that lack surface pockets for interaction, while reliance on a limited number of hotspot residues constraints computational approaches to a narrow range of potential binding modes.

### SUMMARY

A method for protein generation includes determining a binding protein that binds with a target protein using a hybrid diffusion model that jointly generates a discrete residue sequence and a continuous backbone structure for the binding protein. The binding protein is manufactured

A system for protein generation includes a hardware processor and a memory that stores a computer program. When executed by the hardware processor, the computer program causes the hardware processor to determine a binding protein that binds with a target protein using a hybrid diffusion model that jointly generates a discrete residue sequence and a continuous backbone structure for the binding protein and to manufacture the binding protein.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram illustrating the configuration and use of a hybrid diffusion model to generate binding proteins for a target segment, in accordance with an embodiment of the present invention;

FIG. 2 is a block diagram illustrating an architecture of the hybrid diffusion model, in accordance with an embodiment of the present invention;

FIG. 3 is a block/flow diagram of a method of generating binding proteins for a target protein, in accordance with an embodiment of the present invention;

FIG. 4 is a block diagram illustrating a healthcare facility where binding protein generation can be used to create targeted therapies for patients, in accordance with an embodiment of the present invention;

FIG. 5 is a block diagram of a computing device that can be used to generate protein sequences using a hybrid diffusion model, in accordance with an embodiment of the present invention;

FIG. 6 is a diagram showing an exemplary neural network architecture that can be used to implement part of a hybrid diffusion model, in accordance with an embodiment of the present invention; and

FIG. 7 is a diagram showing an exemplary deep neural network architecture that can be used to implement part of a hybrid diffusion model, in accordance with an embodiment of the present invention.

### DETAILED DESCRIPTION OF PREFERRED EMBODIMENTS

A hybrid deep neural network-based diffusion model may be used to jointly design the sequence and structure of binders for arbitrary protein targets in a non-autoregressive manner. The model may include interleaved self-attention layers to capture global correlations among amino acids. The model may further include k-nearest neighbor-based equivariant graph convolutional layers to model local interactions among neighboring residues in three-dimensional space as well as a causal attention layer to mitigate multimodality.

During operation of the mode, protein targets and binders are represented as residue point sets in three-dimensional space, with each residue being associated with a three-dimensional Cartesian coordinate. A diffusion process is used for both continuous residue coordinates and discrete residue types, where noise is progressively added. A joint generative model is trained using a hybrid deep neural network.

By jointly modeling both sequence and structure, as well as their complex interdependencies, the model is able to reduce the primary failure modes in binder design. The enhanced diversity and novelty introduced by the diffusion process contribute to an improved success rate in generating functional binder proteins.

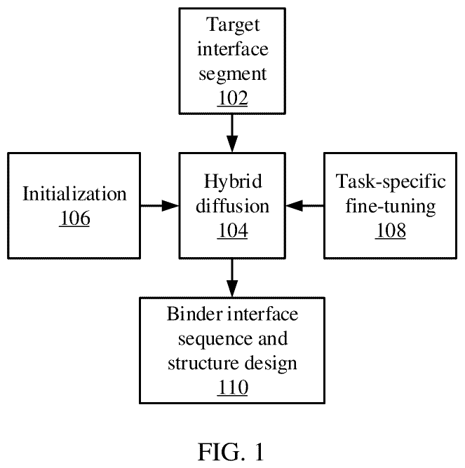

Referring now to FIG. 1, an exemplary protein design flow is shown. A target interface segment 102 is used as input to a hybrid diffusion model 104. As used herein, “hybrid” means that the model generates two modalities, including a protein's sequence and structure expressed as three-dimensional coordinates of each of its residues. Both modalities inform each other through interleaving graph neural network (GNN) and transformer blocks. The hybrid diffusion model 104 has been fine-tuned 108 for the task of protein design, and the diffusion process starts with initialization 106. Task-specific fine tuning 108 may be performed on specific, calibrated protein-protein interaction interface data with the same training scheme as used in pre-training. The output of the model 104 is a binder interface sequence and structure design 110 for a protein.

Initialization 106 may include plane-based initialization or k-nearest-neighbor-based initialization. In k-nearest-neighbor initialization, a random three-dimensional coordinate set is sampled from a random starting point on or close to the target. For each random sample a heuristic interaction energy is estimated based on a k-nearest-neighbor graph and an option having the lowest energy is selected as the initialization.

In plane-based initialization, the target coordinates are used to determine a best-fit flat surface. The geometric center of the target is computed and, from this centroid, an initial starting point is determined by moving a fixed, pre-specified offset distance in a direction perpendicular to the fitted plane, defined by the plane's normal vector. The same initialization is performed from this starting point using the same kNN-based selection method.

A protein includes a chain of amino acids connected by peptide bonds, which folds into a three dimensional structure. The set [symbol] includes the twenty common amino acids. The goal is to generate a binder protein [symbol] conditioned on a target protein [symbol] , where

$${s}^{ℬ} = \{ {s}_{1}^{ℬ} , ... , {s}_{N}^{ℬ} \} and {s}^{𝒯} = \{ {s}_{1}^{𝒯} , ... , {s}_{M}^{𝒯} \}$$

denote the sequences of the binder and target proteins, respectively. The corresponding C_{α}-based backbone structures are represented as

$${x}^{ℬ} = {[ {x}_{1}^{ℬ} , ... , {x}_{N}^{ℬ} ]}^{T} ∈ {ℝ}^{N \times 3} and$$

$${x}^{𝒯} = {[ {x}_{1}^{𝒯} , ... , {x}_{M}^{𝒯} ]}^{T} ∈ {ℝ}^{M \times 3} ,$$

while N and M denote the lengths of the binder and target protein sequences, respectively. The hybrid diffusion model 104 is trained with probability p_{θ}( [symbol] ), where θ indicates the model parameters.

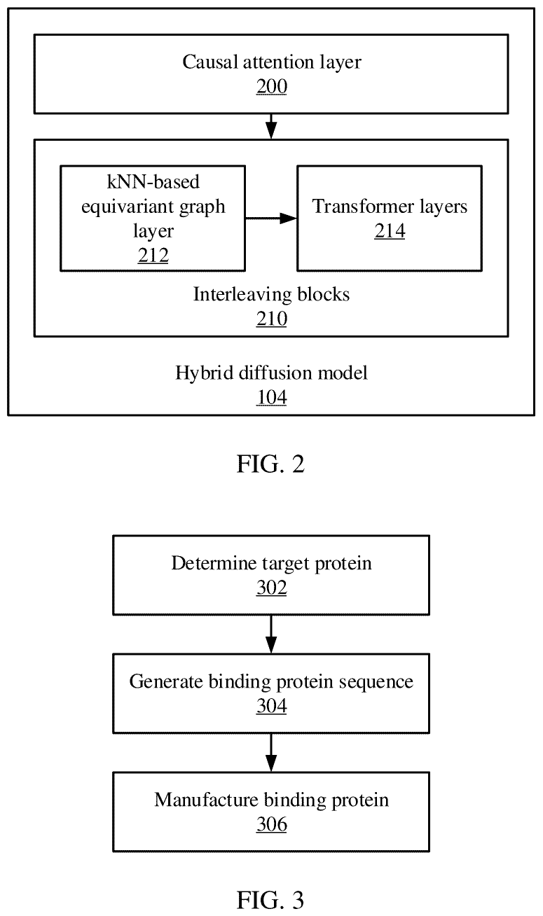

Referring now to FIG. 2, additional detail is shown on the hybrid diffusion model 104. The model 104 generates a sequence and backbone structure of a binder protein for a specific protein target. It may be implemented as a latent variable model. The framework includes a forward diffusion process and a reverse generative process, both defined as Markov chains. The diffusion process gradually perturbs the ground truth data [symbol] into a stationary distribution [symbol] ~qₙₒᵢₛₑ with T increasingly noisy steps:

$$q ( {ℬ}_{1 : T} \vert {ℬ}_{0} , 𝒯 ) = {\prod }_{t = 1}^{T} q ( {ℬ}_{t} \vert {ℬ}_{t - 1} , 𝒯 )$$

To fit the model p_{θ}( [symbol] ) to the data distribution q( [symbol] ), the denoising model may be optimized by the variational bound of the log-likelihood:

$${E}_{q ( {ℬ}_{0} \vert 𝒯 )} [ log {p}_{θ} ( {ℬ}_{0} \vert 𝒯 ) ] \geq {E}_{q ( {ℬ}_{0 : T} \vert 𝒯 )} [ log \frac{{p}_{θ} ( {ℬ}_{0 : T} \vert 𝒯 )}{q ( {ℬ}_{1 : T} \vert {ℬ}_{0} , 𝒯 )} ] = {E}_{q ( {ℬ}_{0} \vert 𝒯 )} [ log {p}_{θ} ( {ℬ}_{0} \vert {ℬ}_{1} , 𝒯 ) + const + {{\sum }_{t = 2}}^{T} {\underbrace{- KL [ q ( {ℬ}_{t - 1} \vert {ℬ}_{t} , {ℬ}_{0} , 𝒯 ) \vert \vert {p}_{θ} ( {ℬ}_{t - 1} \vert {ℬ}_{t} , 𝒯 ) ]}}_{{ε}_{t}} ]$$

To enhance the consistency between the designed binder's sequence and structure, the model 104 is designed to jointly generate both the discrete residue sequence and the continuous backbone structure. Diffusion and generative processes are therefore implemented that operate on both discrete residue sequences and continuous spatial coordinates.

The joint distribution of protein sequences and backbone structures are modeled during the diffusion process as the product of two independent distributions. This formulation allows for efficient sampling of noisy data for both components. For the discrete protein sequence s^{B}, a categorical distribution Cat(s^{B}; p) is used, where p represents a vector on the (| [symbol] |−1)-dimensional probability simplex. For the continuous backbone structure, a Gaussian distribution [symbol] is used to model spatial coordinates. At each diffusion step t, uniform noise is added to the residue types across all categories, and Gaussian noise is applied to the structural coordinates. This process follows a Markov chain with a predefined schedule β₁, β₂, . . . , β_{T} as follows:

$$q ( {ℬ}_{t} \vert {ℬ}_{t - 1} , 𝒯 ) = q ( {s}_{t}^{ℬ} \vert {s}_{t - 1}^{ℬ} , 𝒯 ) \cdot q ( {x}_{t}^{ℬ} \vert {x}_{t - 1}^{ℬ} , 𝒯 )$$

$$q ( {s}_{t}^{ℬ} \vert {s}_{t - 1}^{ℬ} , 𝒯 ) = Cat ( {s}_{t}^{ℬ} ; ( 1 - {β}_{t} ) {s}_{t - 1}^{ℬ} + \frac{{β}_{t}}{K} )$$

$$q ( {x}_{t}^{ℬ} \vert {x}_{t - 1}^{ℬ} , 𝒯 ) = 𝒩 ( {x}_{t}^{ℬ} ; \sqrt{1 - {β}_{t}} {x}_{t - 1}^{ℬ} , {β}_{t} I )$$

In practice, the schedules for different data modalities can be different.

Denoting αₜ=1−βₜ and

$${\overline{α}}_{t} = {\prod }_{i = 1}^{t} {α}_{i} ,$$

the noisy data distributions

$$q ( {s}_{t}^{ℬ} \vert {s}_{0}^{ℬ} , 𝒯 ) and q ( {x}_{t}^{ℬ} \vert {x}_{0}^{ℬ} , 𝒯 )$$

can be calculated in closed form:

$$q ( {s}_{t}^{B} \vert {s}_{0}^{B} , 𝒯 ) = Cat ( {s}_{t}^{B} ; {\overline{α}}_{t} {s}_{0}^{B} + ( 1 - {\overline{α}}_{t} ) / K )$$

$$q ( {x}_{t}^{B} \vert {x}_{0}^{B} , 𝒯 ) = 𝒩 ( {x}_{t}^{B} ; \sqrt{{\overline{α}}_{t}} {x}_{0}^{B} , ( 1 - {\overline{α}}_{t} ) I )$$

The posterior distributions

$$q ( {s}_{t - 1}^{B} \vert {s}_{t}^{B} , {s}_{0}^{B} , 𝒯 ) and q ( {x}_{t - 1}^{B} \vert {x}_{t}^{B} , {x}_{0}^{B} , 𝒯 )$$

using Bayes rule in closed-form:

$$q ( {s}_{t - 1}^{B} \vert {s}_{t}^{B} , {s}_{0}^{B} , 𝒯 ) = Cat ( {s}_{t - 1}^{B} ; {θ}_{post}^{s} ( {s}_{t}^{B} , {s}_{0}^{B} ) )$$

$${θ}_{post}^{s} ( {s}_{t}^{B} , {s}_{0}^{B} ) = {\tilde{θ}}^{s} / {{\sum }_{k = 1}}^{K} {\tilde{θ}}_{k}^{s}$$

$${\tilde{θ}}^{s} = [ {α}_{t} {s}_{t}^{B} + ( 1 - {α}_{t} ) / K ] ⊙ [ {\overline{α}}_{t - 1} {s}_{0}^{B} + ( 1 - {\overline{α}}_{t - 1} ) / K ]$$

$$q ( {x}_{t - 1}^{B} \vert {x}_{t}^{B} , {x}_{0}^{B} , 𝒯 ) = 𝒩 ( {x}_{t - 1}^{B} ; {\tilde{μ}}_{t} ( {x}_{t}^{B} , {x}_{0}^{B} ) , {\tilde{β}}_{t} I )$$

$${\overline{μ}}_{t} ( {x}_{t}^{B} , {x}_{0}^{B} ) = \frac{\sqrt{{\overline{α}}_{t - 1}} {β}_{t}}{1 - {\overline{α}}_{t}} {x}_{0}^{B} + \frac{\sqrt{{\overline{α}}_{t}} ( 1 - {\overline{α}}_{t - 1} )}{1 - {\overline{α}}_{t}} {x}_{t}^{B}$$

$${\tilde{β}}_{t} = \frac{1 - {\overline{α}}_{t - 1}}{1 - {\overline{α}}_{t}} {β}_{t}$$

The generative process recovers the data distribution from the noise distribution. The reverse generative process may be parameterized using a hybrid neural network by θ:

$${p}_{θ} ( {ℬ}_{t - 1} \vert {ℬ}_{t} , 𝒯 ) = {p}_{θ} ( {s}_{t - 1}^{ℬ} \vert {s}_{t}^{ℬ} , 𝒯 ) \cdot {p}_{θ} ( {x}_{t - 1}^{ℬ} \vert {x}_{t}^{ℬ} , 𝒯 )$$

$${p}_{θ} ( {s}_{t - 1}^{ℬ} \vert {s}_{t}^{ℬ} , 𝒯 ) = Cat ( {s}_{t - 1}^{ℬ} ; {θ}_{post}^{s} ( {s}_{t}^{ℬ} , {\hat{s}}_{0}^{ℬ} ) )$$

$${p}_{θ} ( {x}_{t - 1}^{ℬ} \vert {x}_{t}^{ℬ} , 𝒯 ) = 𝒩 ( {x}_{t - 1}^{ℬ} ; {μ}_{θ} ( {s}_{t}^{ℬ} , {x}_{t}^{ℬ} , t , 𝒯 ) , {σ}_{t}^{2} I )$$

The hybrid neural network may composed L_{I} interleaved blocks 210, each containing Lₜᵣₐₙₛ self-attention transformer layers 214 to capture global correlations among amino acids and a kNN-based equivariant graph convolutional layer 212 to model local interactions among neighboring residues in 3D space. In the l-th interleaved block 210, the structure is defined as follows:

$$\begin{matrix}{H}_{t}^{l + 0.5} = Transformer - block ( {H}_{t}^{l} ; 𝒯 ) \\ {m}_{t , ik}^{l + 0.5} = {f}_{m} ( [ {h}_{t , t}^{l + 0.5} ; {h}_{t , k}^{l + 0.5} ; ‖ {x}_{t , t}^{l} - {x}_{t , k}^{l} {‖}_{2} ] ; 𝒯 ) \\ {w}_{t , k}^{l + 0.5} = Soft max ( {m}_{t , ik}^{l + 0.5} ) , {m}_{t , ik}^{l + 1} = {w}_{t . ik ,}^{l + 0.5} \cdot {m}_{t , ik}^{l + 0.5} \\ {c}_{i}^{l + 1} = {\sum }_{k ∈ N ( \dot{l} )} {m}_{t , ik}^{, l + 1} , weight = σ ( F F N ( {c}_{i}^{l + 1} ) ) \\ {h}_{t , i}^{l + 1} = {h}_{t , i}^{l + 0.5} + {\sum }_{k ∈ N ( \dot{l} )} weight \cdot {m}_{t , ik}^{l + 1} \\ {x}_{t , i}^{l + 1} = {x}_{t , i}^{l} + {\sum }_{k ∈ N ( l )} ( {x}_{t , i}^{l} - {x}_{t , k}^{l} ) \cdot {f}_{x} ( {m}_{t , ik}^{l + 1} )\end{matrix}$$

where

$${H}_{t}^{l} = {[ {h}_{t , {1}^{,}}^{l} ′ {h}_{t , 2}^{l} , … , {h}_{t , N}^{l} ]}^{T}$$

denotes the residue representation matrix at l-th block and time step t, while

$${H}_{t}^{0} = {[ {s}_{1}^{ℬ} , {s}_{2}^{ℬ} , … , {s}_{N}^{ℬ} ]}^{T}$$

is the one-hot embedding matrix of binder protein sequence. Functions fₘ, fₓ and FFN denote feed-forward layers and σ denotes sigmoid function. N(i) denotes the set of k-nearest neighbors of the i-th residue. Finally,

$${x}_{t}^{{L}_{1}}$$

is the predicted structure at time step t, i.e.

$${\hat{x}}_{0}^{B}$$

To address the multimodality issue, a causal attention layer 200 is added on top of the hybrid neural network 104:

$$\begin{matrix}{\tilde{h}}_{t , i}^{{L}_{I}} = L a y e r N o r m ( M H A ( {h}_{t , i}^{{L}_{I}} , {H}_{t , 1 : i}^{{L}_{1}} ) + {h}_{t , i}^{{L}_{I}} ) \\ {h}_{t , i}^{out} = Laye r N orm ( F F N ( {\overline{h}}_{t , i}^{{L}_{1}} ) + {\overline{h}}_{t , i}^{{L}_{1}} ) \\ {\hat{\hat{s}}}_{0}^{ℬ} = Soft max ( {H}_{t}^{out} )\end{matrix}$$

where MHA denotes multi-head attention and the i-th residue can only attend to its previous residues.

$${H}_{t , 1 : i}^{{L}_{1}} .$$

The posterior distributions

$${p}_{θ} ( {s}_{t - 1}^{ℬ} \vert {s}_{t}^{ℬ} , 𝒯 ) {p}_{θ} ( {x}_{t - 1}^{ℬ} \vert {x}_{t}^{ℬ} , 𝒯 )$$

can be approximated using the predicted

$${\hat{s}}_{0}^{ℬ} and {\hat{x}}_{0}^{ℬ} .$$

The model 104 may be trained by maximizing the variational lower bound of the log-likelihood, where [symbol] is calculated as:

$$\begin{matrix}{ℒ}_{t} = - KL [ q ( {s}_{t - 1}^{B} \vert {s}_{t}^{B} , {s}_{0}^{B} , 𝒯 ) ‖ {p}_{θ} ( {s}_{t - 1}^{E} \vert {s}_{t}^{B} , 𝒯 ) ] \\ - KL [ q ( {x}_{t - 1}^{B} \vert {x}_{t}^{B} , {x}_{0}^{B} , 𝒯 ) ‖ {p}_{θ} ( {x}_{t - 1}^{B} 1 {x}_{t}^{B} , 𝒯 ) ] \\ = \begin{matrix}\underbrace{- \{ {\sum }_{k} {{θ}_{p o s t}^{x} ( {s}_{t}^{B} , {s}_{0}^{B} )}_{k} log \frac{{{θ}_{p o s t}^{s} ( {s}_{c}^{ℬ ,} {s}_{0}^{B} )}_{k}}{{{θ}_{p o s t}^{s} ( {s}_{t}^{B} , {s}_{k} )}_{k}} \}} \\ {ℒ}_{t}^{B}\end{matrix} \\ \begin{matrix}\underbrace{- \{ \frac{1}{2 {σ}_{c}^{2}} ‖ {\tilde{μ}}_{c} ( {x}_{c}^{B} , {x}_{0}^{B} ) - {μ}_{θ} ( {s}_{c}^{B} , {x}_{c}^{B} , t , 𝒯 ) {‖}^{2} + c o n s t \}} \\ {ℒ}_{t}^{a}\end{matrix}\end{matrix}$$

$${ℒ}_{t}^{x}$$

can be further simplified as:

$${ℒ}_{t}^{x} = - \{ {λ}_{t} ‖ {x}_{0}^{ℬ} - {\hat{x}}_{0}^{ℬ} {‖}^{2} + c o n s t \} , {λ}_{t} = \frac{{\overline{α}}_{t - 1} {β}_{t}^{2}}{2 {{σ}^{2} ( 1 - {\overline{α}}_{t} )}^{2}}$$

with λₜ being set to 1. Furthermore, log p_{θ}( [symbol] ) can be calculated as:

$$\begin{matrix}log {p}_{θ} ( {ℬ}_{0} \vert {ℬ}_{1} , 𝒯 ) = log {p}_{θ} ( {s}_{0}^{ℬ} \vert {s}_{1}^{ℰ} , 𝒯 ) + \\ log {p}_{θ} ( {x}_{0}^{ℰ} 1 {x}_{1}^{ℬ} , 𝒯 ) \\ = {\sum }_{k} {s}_{0 , k}^{ℬ} log {\hat{\hat{s}}}_{0 ,}^{ℬ} + ‖ {x}_{0}^{ℰ} - {\hat{x}}_{0}^{ℰ} {‖}^{2}\end{matrix}$$

An informative prior distribution can significantly enhance model performance. To this end, an informative filter may be used to select better initial model inputs rather than relying on random noise. Specifically, for backbone structure initialization, the average distance may be computed between each residue and its k-nearest neighbors to define the following energy function:

$${E}_{k n n} ( ℬ ) = {\sum }_{i = 1}^{N} {( k n n d i s t ( {x}_{i} ) - {μ}_{k n n} )}^{2}$$

where

$$knndist ( {x}_{i} ) = \frac{1}{k} {Σ}_{j ∈ N ( i )} ‖ {x}_{i} - {x}_{j} {‖}^{2}$$

denotes the average distance from the i^{th} residue to its k nearest neighbors and μₖₙₙ is the empirical mean of knndist(x) across all residues in the training dataset. In some embodiments, k may be set to 4. For sequence initialization, secondary structure fragments collected from the training dataset may be sampled. This ensures that the initialized structures and sequences exhibit geometric similarities to the training data, providing the model with an informative starting point for candidate generation.

Referring now to FIG. 3, a method of manufacturing binding proteins is shown. Block 302 identifies a target protein, which for example may include a protein sequence that is present on a pathogen or cancer cell. Block 304 generates a binding protein sequence using the hybrid diffusion model 104. Block 306 then manufactures the binding protein, for example as part of a targeted pharmaceutical or vaccine.

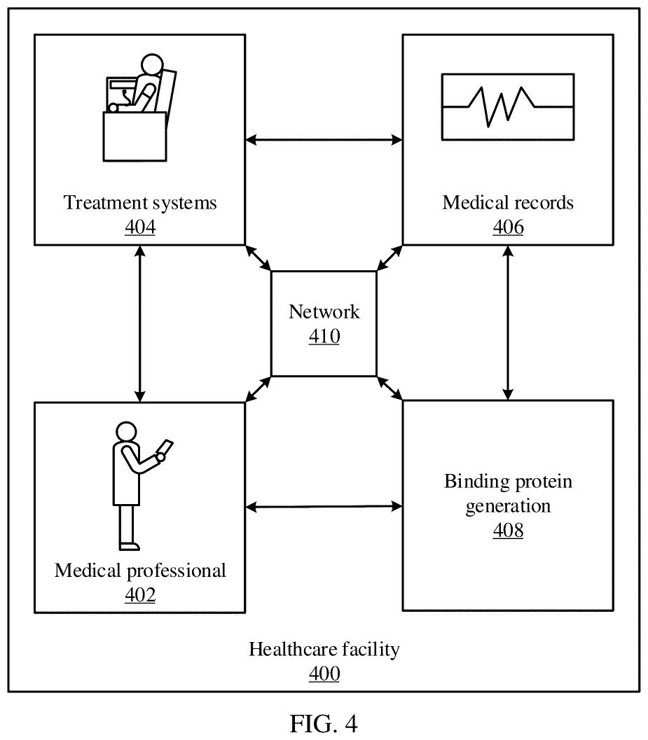

Referring now to FIG. 4, a diagram of time series analysis is shown in the context of a healthcare facility 400. Binding protein generation 408 may be used to create new therapies and pharmaceuticals, for example targeted to a specific patient or illness. The binding protein generation 408 may identify a protein that binds to a target protein, for example one identified as part of a specific pathogen or cancer cell.

The healthcare facility may include one or more medical professionals 402 who review information extracted from a patient's medical records 406 to determine their healthcare and treatment needs. These medical records 406 may include self-reported information from the patient, test results, and notes by healthcare personnel made to the patient's file. Treatment systems 404 may furthermore monitor patient status to generate medical records 406 and may be designed to automatically administer and adjust treatments as needed.

The different elements of the healthcare facility 400 may communicate with one another via a network 410, for example using any appropriate wired or wireless communications protocol and medium. Thus the binding protein generation 408 receives information about a target protein sequence data from treatment systems 404, medical professionals 402, and from medical records 406, and generates binding proteins that are tailored to the target protein sequence. The medical professionals 402 may coordinate treatment based on the generated proteins, and in some cases a treatment may be automatically administered or altered based on these proteins.

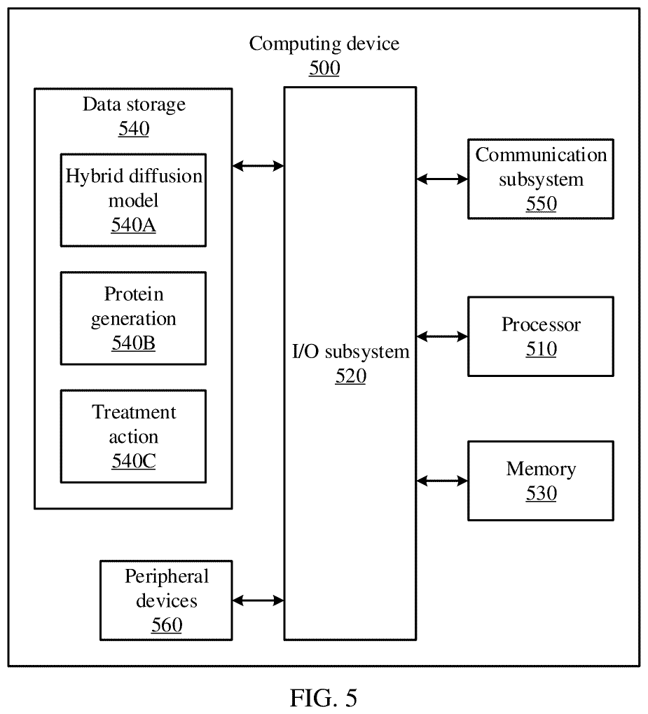

Referring now to FIG. 5, an exemplary computing device 500 is shown, in accordance with an embodiment of the present invention. The computing device 500 is configured to generate binding proteins.

The computing device 500 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 500 may be embodied as one or more compute sleds, memory sleds, or other racks, sleds, computing chassis, or other components of a physically disaggregated computing device.

As shown in FIG. 5, the computing device 500 illustratively includes the processor 510, an input/output subsystem 520, a memory 530, a data storage device 540, and a communication subsystem 550, and/or other components and devices commonly found in a server or similar computing device. The computing device 500 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 530, or portions thereof, may be incorporated in the processor 510 in some embodiments.

The processor 510 may be embodied as any type of processor capable of performing the functions described herein. The processor 510 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 530 may be embodied as any type of volatile or non-volatile memory or data storage capable of performing the functions described herein. In operation, the memory 530 may store various data and software used during operation of the computing device 500, such as operating systems, applications, programs, libraries, and drivers. The memory 530 is communicatively coupled to the processor 510 via the I/O subsystem 520, which may be embodied as circuitry and/or components to facilitate input/output operations with the processor 510, the memory 530, and other components of the computing device 500. For example, the I/O subsystem 520 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 520 may form a portion of a system-on-a-chip (SOC) and be incorporated, along with the processor 510, the memory 530, and other components of the computing device 500, on a single integrated circuit chip.

The data storage device 540 may be embodied as any type of device or devices configured for short-term or long-term storage of data such as, for example, memory devices and circuits, memory cards, hard disk drives, solid state drives, or other data storage devices. The data storage device 540 can store program code 540A for a hybrid diffusion model, 540B for generating binding proteins, and/or 540C for performing treatment actions. Any or all of these program code blocks may be included in a given computing system. The communication subsystem 550 of the computing device 500 may be embodied as any network interface controller or other communication circuit, device, or collection thereof, capable of enabling communications between the computing device 500 and other remote devices over a network. The communication subsystem 550 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 500 may also include one or more peripheral devices 560. The peripheral devices 560 may include any number of additional input/output devices, interface devices, and/or other peripheral devices. For example, in some embodiments, the peripheral devices 560 may include a display, touch screen, graphics circuitry, keyboard, mouse, speaker system, microphone, network interface, and/or other input/output devices, interface devices, and/or peripheral devices.

Of course, the computing device 500 may also include other elements (not shown), as readily contemplated by one of skill in the art, as well as omit certain elements. For example, various other sensors, input devices, and/or output devices can be included in computing device 500, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 500 are readily contemplated by one of ordinary skill in the art given the teachings of the present invention provided herein.

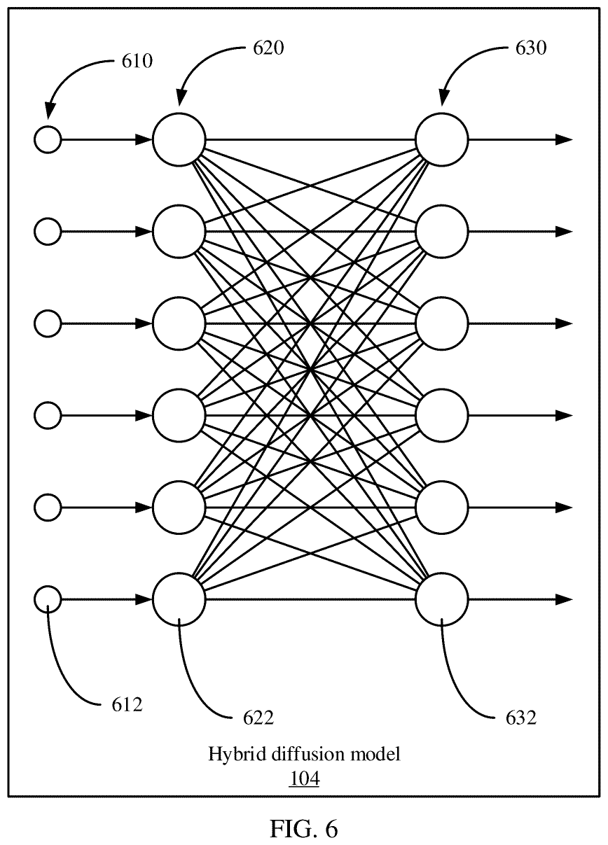

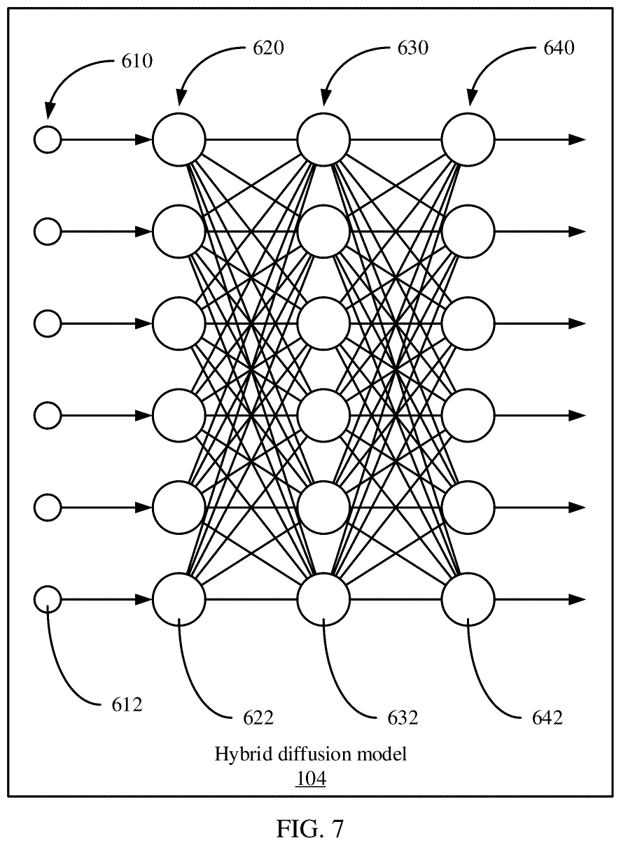

Referring now to FIGS. 6 and 7, exemplary neural network architectures are shown, which may be used to implement parts of the present machine learning models, such as the hybrid diffusion model 104. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 620 of source nodes 622, and a single computation layer 630 having one or more computation nodes 632 that also act as output nodes, where there is a single computation node 632 for each possible category into which the input example could be classified. An input layer 620 can have a number of source nodes 622 equal to the number of data values 612 in the input data 610. The data values 612 in the input data 610 can be represented as a column vector. Each computation node 632 in the computation layer 630 generates a linear combination of weighted values from the input data 610 fed into input nodes 620, and applies a non-linear activation function that is differentiable to the sum. The exemplary simple neural network can perform classification on linearly separable examples (e.g., patterns).

A deep neural network, such as a multilayer perceptron, can have an input layer 620 of source nodes 622, one or more computation layer(s) 630 having one or more computation nodes 632, and an output layer 640, where there is a single output node 642 for each possible category into which the input example could be classified. An input layer 620 can have a number of source nodes 622 equal to the number of data values 612 in the input data 610. The computation nodes 632 in the computation layer(s) 630 can also be referred to as hidden layers, because they are between the source nodes 622 and output node(s) 642 and are not directly observed. Each node 632, 642 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 632 in the one or more computation (hidden) layer(s) 630 perform a nonlinear transformation on the input data 612 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for protein generation, comprising:
determining a binding protein that binds with a target protein using a hybrid diffusion model that jointly generates a discrete residue sequence and a continuous backbone structure for the binding protein; and
manufacturing the binding protein.

2. The method of claim 1, wherein the hybrid diffusion model includes a causal attention layer and a plurality of interleaving blocks.

3. The method of claim 2, wherein each of the plurality of interleaving blocks includes a k-nearest neighbor-based equivariant graph layer and a plurality of transformer layers.

4. The method of claim 3, wherein the plurality of transformer layers capture global correlations among amino acids.

5. The method of claim 3, wherein the k-nearest neighbor-based equivariant graph layers model local interactions among neighboring residues in three-dimensional space.

6. The method of claim 2, wherein the causal attention layer includes a multi-head attention where a given residue can only attend to its previous residues.

7. The method of claim 1, wherein determining the binding protein includes initializing a protein using plane-based initialization.

8. The method of claim 1, wherein determining the binding protein includes initializing a protein using k-nearest-neighbor initialization.

9. The method of claim 1, wherein the target protein is associated with a pathogen or cancer.

10. The method of claim 9, further comprising administering the binding protein to a patient to treat the pathogen or cancer.

11. A system for protein generation, comprising:
a hardware processor; and
a memory that stores a computer program which, when executed by the hardware processor, causes the hardware processor to:
determine a binding protein that binds with a target protein using a hybrid diffusion model that jointly generates a discrete residue sequence and a continuous backbone structure for the binding protein; and
manufacture the binding protein.

12. The system of claim 11, wherein the hybrid diffusion model includes a causal attention layer and a plurality of interleaving blocks.

13. The system of claim 12, wherein each of the plurality of interleaving blocks includes a k-nearest neighbor-based equivariant graph layer and a plurality of transformer layers.

14. The system of claim 13, wherein the plurality of transformer layers capture global correlations among amino acids.

15. The system of claim 13, wherein the k-nearest neighbor-based equivariant graph layers model local interactions among neighboring residues in three-dimensional space.

16. The system of claim 12, wherein the causal attention layer includes a multi-head attention where a given residue can only attend to its previous residues.

17. The system of claim 11, wherein determining the binding protein includes initializing a protein using plane-based initialization.

18. The system of claim 11, wherein determining the binding protein includes initializing a protein using k-nearest-neighbor initialization.

19. The system of claim 11, wherein the target protein is associated with a pathogen or cancer.

20. The system of claim 19, further comprising administering the binding protein to a patient to treat the pathogen or cancer.
